(19) United States
(12) Patent Application Publication
(10) Pub. No.: US 2007/0282363 A1
(43) Pub. Date: Dec. 6, 2007

(54) Annular Cutter For Body Organs

(76) Inventor: Pepi Dakov, Rego Park, NY (US)

Correspondence Address: Pepi Dakov, 62-60 99th Street, Apt 1227, Rego Park, NJ, 11374, US

(21) Appl. No.: 11/660,487

(22) PCT Filed: Aug. 15, 2005
(86) PCT No.: PCT/US05/28883

§ 371 Date: Feb. 16, 2007

Related U.S. Application Data
(60) Provisional application No. 60/602,352, filed on Aug. 18, 2004.

(51) Int. Cl. A61B 17/34

(52) U.S. Cl. 606/185

(57) ABSTRACT

An annular cutter comprising a tubular body with a cutting blade rotational around a central axis, and a finger slot at the upper end of the axis. The cutting blade cuts out a portion of a body organ as the tubular body is turned repeatedly while it is kept in stable position by a finger inserted into the slot at the upper end of the cutter. Means retaining the cutout portion of the body organ are further provided.

### TECHNICAL FIELD

This invention pertains to devices for cutting and removing an annular portion out of body organs. More particularly, this invention pertains to devices for cutting an annular opening in hollow body organs (such as the aorta) for anastomosing purposes (such as coronary artery bypass grafting).

Aortic punches have been used for decades to perform an opening in the aorta to which a graft is sutured and then the graft is joined to a coronary artery. A traditional aortic punch cuts the aortic wall between an anvil and an annular blade.

First, an incision is made with a scalpel and the anvil is inserted into the aorta. After that the device is actuated in a syringe like manner and the wall of the aorta is pressed and cut between an anvil and an annular blade. Cutting by applying a force and movement in axial direction only crushes the tissues and creates an opening with irregular shape and frayed edges. Newer aortic punchers add a rotary motion to the action of the axially moving blade. However, most of the cutting is still accomplished by axially moving blade which constitutes just a minor enhancement over traditional aortic punchers.

Recently aortic cutters have been developed which cut mostly by a rotary motion.

The problem with these devices is that they require repetitive turning of the rotary blade as much as needed until the resilient and sometimes calcified aortic wall is completely severed. It is inconvenient to hold and turn the instrument repeatedly with one hand. Rotating of the cutting blade can be automated, but this necessarily complicates the design, increases the device cost, and makes it prone to technical failures. In addition, the new aortic cutters do not retain securely the cutout portion, which could lead to thromboembolic complications if the plug falls within the blood flow.

### DISCLOSURE OF INVENTION

### Technical Problem

Aortic punchers have several important drawbacks: they require a preceding incision with a scalpel to insert the anvil, they require a strong hand force, and they cut holes is with irregular shapes and frayed edges. Recent aortic cutters are inconvenient to turn and hold with one hand and they do not retain securely the cutout portion of the aortic wall.

### Technical Solution

The new annular cutter for body organs resolves the issues of prior art devices. It does not require an incision with a scalpel, it cuts with a minimal hand force, and it cuts out perfectly shaped holes with smooth edges. It can be easily hold and turned with one hand.

The annular cutter has a tubular axially elongated body. An annular cutting blade is affixed to the lower end of the axial body. The upper end of the cutter has a ring into which a fingertip is inserted. The cutting blade cuts out a hole as the tubular body is turned repeatedly while the cutter is stabilized with the index finger inserted into the finger slot on the upper end. The cutout portion is securely retained by a pointed barbed end.

### Advantageous Effects

The novel annular cutter for body organs offer surgeons several important advantages compared to currently used devices: it does not require a preceding scalpel incision, which shortens the procedure; it cuts easy with a minimal hand force; it creates a perfectly round hole with smooth edges, it can be easily operated with one hand, and it reduces the risk of thromboembolic complications. The cutter has a simple structure and method of operation, which minimizes the risk of technical failure and makes it easy and inexpensive to manufacture.

### DESCRIPTION OF DRAWINGS

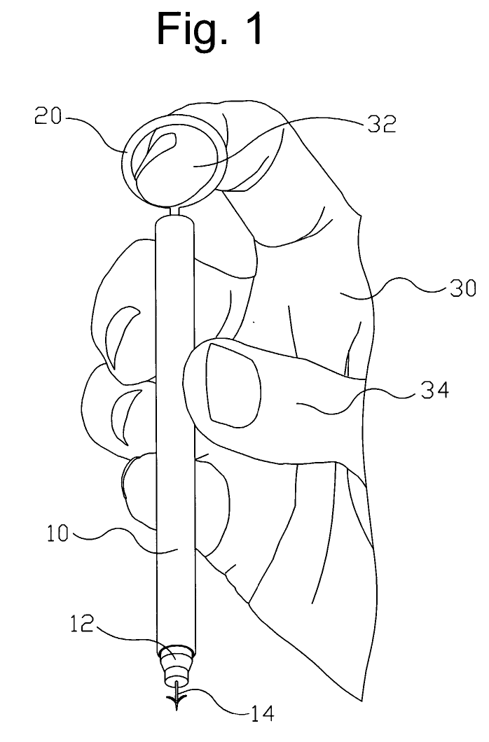

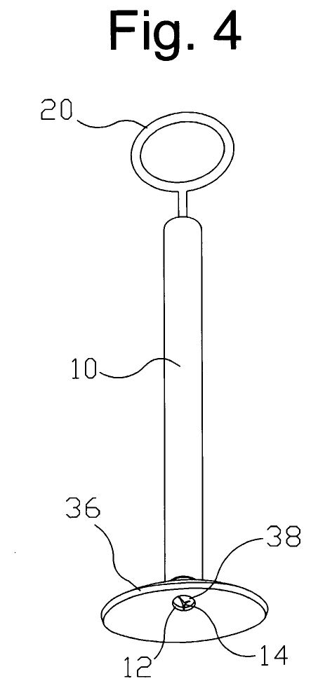

Embodiments of the novel annular cutter for body organs are shown in FIGS. 1 through 8. In general, the cutter consists of an elongated tubular body 10 with a cutting blade 12 at the lower end. The elongated body and cutting blade rotate around a central axis that has a barbed pointed end 14 at the lower end and a ring 20 at the upper end. The cutter is operated with one hand 30 as the cutter is kept in a stable position by the index finger 32 inserted into the ring 20, while the tubular body 10 is rotated between the thumb 34 and the other fingers.

### BEST MODE

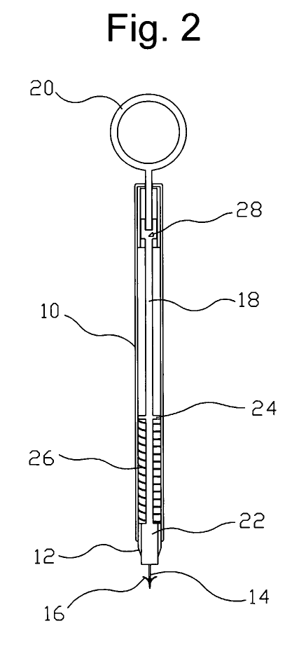

Most preferred embodiment of an annular cutter is illustrated in FIGS. 1 through 3C. The cutter is shown whole and cut in half in prospective views in FIGS. 1 and 2.

The cutter has an elongated tubular body 10. An annular blade 12 is affixed to the lower portion of the elongated body. The elongated body and cutting blade rotate around shaft 18 that is in central axial position. In the lower end, the shaft terminates in a barbed pointed end 14 that has reverse projecting barbs 16. On the upper end, the central shaft ends with a ring 20 that is large enough to accommodate the tip of a finger. An elastic spring 26 is engaged between the upper edge of cutting blade and a flange 24 of the shaft. The spring exerts an elastic force that pushes the shaft up which moves up the barbed pointed end 14 towards the cutting blade 12. A turning mechanism 28 turns the shaft and respectively the barbs at approximately 90 degrees when the shaft is moves up. The upper portion of the shaft ends with a ring 20.

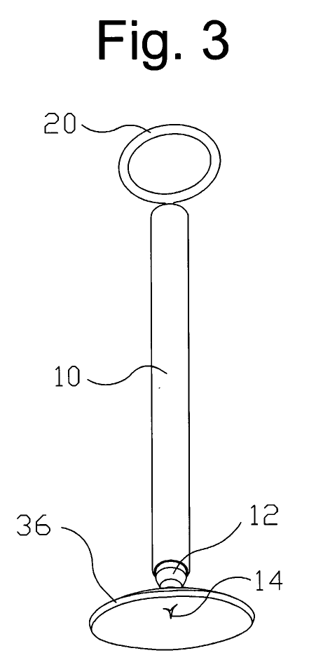

It is easy and quick to cut a hole in a body organ, such as the aorta, with the annular cutter, as this is illustrated in FIGS. 3, 4, and 5. Pushing down with the index finger 32 inserted in the ring 20, which brings out the barbed pointed end 14 in front of the cutting blade 12, the surgeon inserts the barbed pointed end into aorta 36. Then the surgeon relaxes the index finger, which brings the shaft up by the force of elastic spring 14, and starts turning the elongated body with the fingers. During the upward movement of the shaft, the barbed pointed end turns about ninety degrees. In this way the barbs turn and catch the aortic wall in areas different from the place of their insertion.

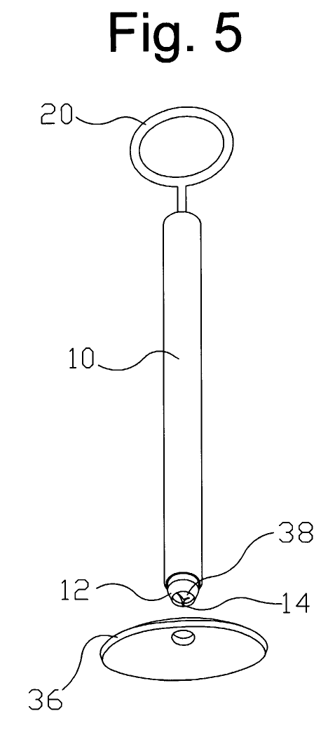

The aortic wall is cut by the repetitive turning of the cutting blade applied under a light pressure on the aortic wall. During this process, the cutter is stabilized in two points on both ends. On the lower end, it is kept in place by the pointed barbed end 14 inserted into the aortic wall 36. On the upper end it is stabilized by the index finger inserted in the ring 20. This enables the surgeon to turn, let go, and turn again the elongated body repeatedly as many times as needed till the entire wall is cut through. In this way, the aortic wall is cut quickly and easily with one hand with minimal hand force. The cutout portion 38, securely retained by the turned barbs, withdraws within the annular blade by the upward force of the spring and the cutter is removed away, as shown in FIG. 5.

### Other Embodiments

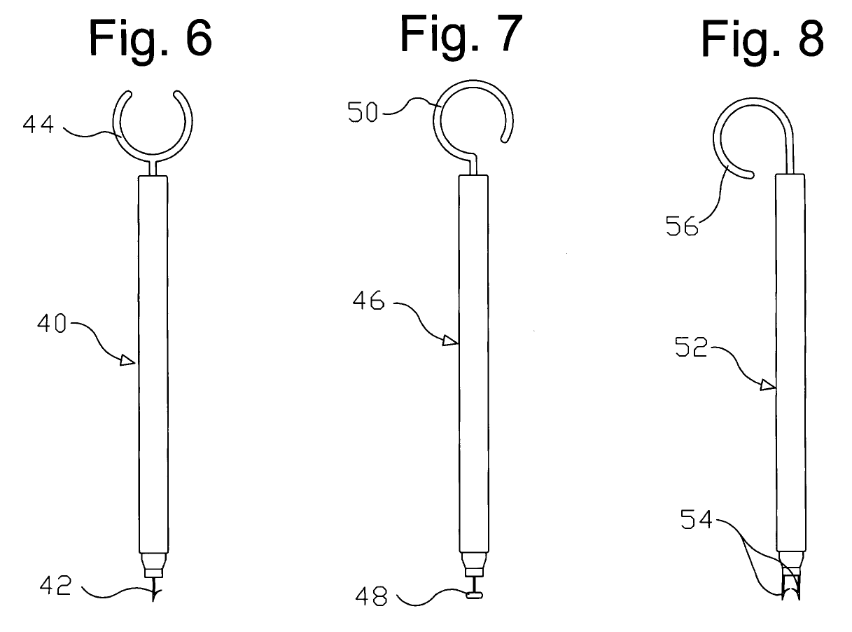

One skilled in the art can build various embodiments based on the annular cutter described above. A specific embodiment of an annular cutter for body organs is shown in FIG. 6. The cutter 40 has a modified lower end 42 with only one barb and a modified upper end with a ring 44 that is open. The open design allows the ring to be narrowed or enlarged accordingly to the size of the finger of the surgeon. Another specific embodiment of an annular cutter for body organs is shown in FIG. 7. The cutter 46 has a modified lower end 48 with an anvil shape and a modified upper end 50 with a hook-shaped finger slot. One additional specific embodiment of an annular cutter for body organs is shown in FIG. 8. The cutter 52 has two pointed barbed ends 54. On the upper end it has an open ring 56 that is sideway of the midline axis, which can further facilitate the handling of the cutter.

Based on the novel annular cutter described above, one skilled in the art can build other embodiments enhancing the functionality of the cutter, some examples of which are: the elongated body can have manufactured with rough or grooved surface to provide a better grip; the ring can be positioned inclined to the axis to permit easer finger insertion; the finger slot can be enlarged and/or arched to the side so the cutter is better stabilized by inserted third and/or fourth fingers, while the index finger takes part in the turning; and the cutting blade can be serrated to improve cutting of harder tissues, such as severely calcified aortic walls.

### INDUSTRIAL APPLICABILITY

The annular cutter for body organs can find implementation in various surgical procedures: to make holes in the walls of hollow organs for anastomosing purposes or for inserting tubes, for cutting and removing tissues of solid organs in tumor cases, for cutting out a hole in dental implants, and in any other cases in which an annular piece of tissue needs to be cut out of a body organ. The annular cutter can be highly beneficial in coronary artery bypass grafting as it improves the work of surgeons in several aspects: it eases the work of the surgeon, it speeds the procedure, and it safely cuts out a perfectly shaped hole with smooth edges.

1. An annular cutter for body organs comprising: a cutting blade at a lower end of a tubular body rotational around a central axis; a finger slot extending from upper end of said central axis and retaining means extending from lower end of said axis, wherein a finger inserted in said slot stabilizes turning of said tubular body and cutting with said cutting blade of an annular portion out of a body organ held by said retaining means inserted into said portion of said body organ.

2. Wherein said annular cutter of claim 1 further comprising a finger slot with a shape of a ring.

3. Wherein said annular cutter of claim 2 further comprising a finger slot with a shape of an open ring.

4. Wherein said retaining means of claim 1 further comprising a pointed end with reverse projecting barbs.

5. Wherein said annular cutter of claim 4 further comprising an elastic spring engaged to move up said retaining means towards said cutting blade.

6. Wherein said annular cutter of claim 5 further comprising means for turning said retaining means when moved up so the turned barbs hold said organ in areas different from their place of insertion.

7. An annular cutter for body organs comprising: a cutting blade affixed to a lower end of a tubular body rotational around a central axis, and a finger slot extending from upper end of said central axis, wherein a finger inserted in said slot stabilizes turning of said annular body and cutting with said cutting blade of an annular portion out of a body organ.

8. Wherein said annular cutter of claim 7 further comprising a finger slot with a shape of a ring.

9. Wherein said annular cutter of claim 8 further comprising a finger slot with a shape of an open ring.

10. Wherein said annular cutter of claim 7 further comprising retaining means that are inserted into said portion of said body organ, said retaining means holding said portion when cut and removed out of said body organ.

11. Wherein said retaining means of claim 10 further comprising a pointed end with barbs.

12. Wherein said annular cutter of claim 11 further comprising an elastic spring engaged to move up said retaining means towards said cutting blade.

13. Wherein said annular cutter of claim 12 further comprising means for turning said retaining means when moved up so the turned barbs catch said portion of said body organ in areas different from their place of insertion.

14. An annular cutter for body organs comprising: a cutting edge in a lower end of a tubular body rotational around a central axis including a slot in upper end of said axis, wherein a finger inserted in said slot stabilizes turning of said tubular body and cutting with said cutting edge of an annular portion out of a body organ.

15. Wherein said annular cutter of claim 14 further comprising a finger slot with a shape of a ring.

16. Wherein said annular cutter of claim 15 further comprising a finger slot with a shape of an open ring.

17. Wherein said central axis of said annular cutter of claim 14 further comprising retaining means that are inserted into said portion of said body organ, said retaining means holding said portion when cut and removed out of said body organ.

18. Wherein said retaining means of claim 17 further comprising a pointed end with barbs.

19. Wherein said annular cutter of claim 18 further comprising an elastic spring engaged to move upward said retaining means towards said cutting edge.

20. Wherein said annular cutter of claim 19 further comprising means for turning said pointed end with barbs when moved up so the turned barbs catch said portion of said organ in areas different from their place of insertion.
